(19) United States
(12) Patent Application Publication
(10) Pub. No.: US 2006/0286547 A1
(43) Pub. Date: Dec. 21, 2006

(54) Time-of-addition assay for identifying anti-viral compounds

(76) Inventors: Koenraad Lodewidjk August Van Acker, Temse (BE); Lieve Elisabeth Louis Bunkens, Vilvoorde (BE); Gery Karel Julia Dams, Paal-Beringen (BE); Kurt Hertogs, Nieuwpoort (BE); Tania Pauline Eduard Ivens, Melsele (BE); Erik Jos Amandus Nijs, Rillaar (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/570,358

(22) PCT Filed: Sep. 20, 2004
(86) PCT No.: PCT/EP04/52250

§ 371 Date: Mar. 1, 2006

Related U.S. Application Data
(60) Provisional application No. 60/567,221, filed on Apr. 30, 2004.

(30) Foreign Application Priority Data
Sep. 19, 2003 (EP) 03103472.1

(51) Int. Cl. A61K 38/00; C12M 1/34; C12Q 1/70

(52) U.S. Cl. 435/5; 435/287.2; 514/2

(57) ABSTRACT

The present invention relates to a multi-well assay for identifying a compound inhibiting the replication cycle of a micro-organism, for example, HIV, and to an apparatus for carrying out an assay according to the present invention. The invention further relates to compounds identifiable with an assay according to the invention and pharmaceutical compositions comprising a therapeutically effective amount of such compounds. The identified compounds may be used as medicaments, and in particular for the manufacture of a medicament for treating infectious diseases and in method for treating infectious diseases including AIDS.

### FIELD OF THE INVENTION

The present invention relates to the medicinal field. In a first aspect, the present invention relates to an assay for identifying compounds inhibiting the replication cycle of a micro-organism e.g. HIV. In another aspect, the invention relates to an apparatus for carrying out the assay according to the invention. The invention further relates to compounds identifiable with an assay according to the invention.

### BACKGROUND

A retrovirus, designated human immunodeficiency virus (HIV), is the etiological agent of the complex disease that includes progressive destruction of the immune system (acquired immune deficiency syndrome; AIDS) and degeneration of the central and peripheral nervous system. This virus is known by different names, including T-lymphocyte virus III (HTLV-III) or lymphadenopathy-associated virus (LAV) or AIDS-related virus (ARV) or human immunodeficiency virus (HIV). Up until now, two distinct families have been identified, i.e. HIV-1 and HIV-2. Hereinafter, HIV will be used to generically denote these viruses.

HIV is a type of retrovirus, belonging to single stranded RNA viruses, that can infect a number of different cells, including CD4 bearing macrophages and T-helper lymphocytes within a host. After initial contact and attachment to a cell of the immune system (e.g. lymphocytes, monocytes), there is a cascade of intracellular events. The end product of these events is the production of massive numbers of new viral particles, death of the infected cells, and ultimate devastation of the immune system.

The HIV replication cycle consists of different stages, which can be broadly identified as a) an entry stage, b) a reverse transcription stage, c) an integration stage, d) a budding stage and d) maturation stage (in which protease plays an important role).

In the entry stage, HIV enters a CD4 cell. This process as such also comprises different phases. In a first phase, a CD4 receptor attachment phase, HIV infection begins with the interaction of the HIV glycoprotein (gp) 120 with the CD4 receptor molecule on the surface of a target cell. In a second phase, a co-receptor binding phase, following CD4 binding, a center material change in the HIV gp120/gp 41 complex is induced by interaction of gp120 with the chemokine receptors CCR5 or CXCR4. This change In confirmation exposes gp 41 allowing it to initiate fusion of the membranes. In a third phase, a phase involving membrane fusion events, as the virus fuses with the cell, internalization of the viral core with the associated RNA occurs. Partial uncoating of the viral core occurs to expose the viral RNA.

Once in the cell cytoplasm, the conversion of the viral RNA into double-stranded DNA commences. In the following replication stage, the viral reverse transcriptase becomes active. Reverse transcriptase synthesizes a double-stranded DNA copy of the single-stranded viral RNA generating a provirus.

Subsequently, in the integration stage, the viral DNA migrates to and enters the host cell nucleus and becomes integrated into the cell DNA with the help of the enzyme integrase. The provirus can then remain latent or be active, generating products for the generation of new virons. Inside the nucleus, RNA polymerase II transcribes viral DNA into mRNA. The viral mRNA leaves the nucleus.

A further stage involves a protease activity stage. When viral RNA is translated into a polypeptide sequence, that sequence is assembled in a long chain that includes several individual proteins (reverse transcriptase, protease, integrase). Before these enzymes become functional, they must be cut from the longer polypeptide chain. Viral protease cuts the long chain into its individual enzyme components, which then facilitates the production of new viruses.

Several of the stages in the above-explained viral replication cycle, may be taken advantage of in developing anti-retroviral therapies. It is known that some antiviral compounds act as inhibitors of HIV on particular stages in the HIV replication cycle and are effective agents in the treatment of HIV and similar diseases. Fusion inhibitors, for instance, work outside the cell to prevent the first stage of HIV replication. They prevent HIV from entering the CD4 cell by blocking fusion of the outer membrane of the virus with the cell membrane. Another major class of drugs found useful in slowing HIV infections are collectively called “reverse transcriptase inhibitors”. These act by blocking the recoding of viral RNA into DNA. The reverse transcription stage of HIV replication provides a good target for the action of two types of drugs: nucleoside reverse transcriptase inhibitors (nucleoside analogs) and non-nucleoside reverse transcriptase Inhibitors (NNRTIs). Also a number of integrase inhibitors have been reported. These include, nucleotide-based inhibitors, known DNA binders, catechols and hydrazide containing derivatives. They prevent the insertion of viral cDNA into the host cell genome, which is a critical stage in the viral life cycle. Protease inhibitors work at the last stage of the viral replication cycle. They prevent HIV from being successfully assembled and released from the Infected CD4 cell. By blocking the ability of the protease to cleave the viral polypeptide into functional enzymes, protease inhibitors interfere with continued Infection.

However, despite the fact that various HIV-inhibitors have been identified that are very useful in the treatment of AIDS, they have a common limitation, namely, the targeted enzymes in the HIV virus are able to mutate in such a way that the known drugs become less effective, or even ineffective against these mutant HIV viruses. Or, in other words, the HIV virus creates an ever-increasing resistance against the available drugs. Also, due to an insufficient drug potency in certain cases, incomplete viral suppression may provide fertile ground for resistance to emerge.

Thus, due to the great propensity of HIV-1 to mutate, and a continuing growth in the worldwide infection rate, the search for additional therapies for AIDS has been of the highest priority. The emergence of human immunodeficiency virus type 1 (HIV-1) strains resistant to highly active antiretroviral therapy necessitates continued drug discovery for the treatment of HIV-1 infection. It is clear that there is a great need in the art for new antiviral agents to reduce the rate of resistance and suppress viral replication even further.

Fenard et al., Journal of Clinical Investigation, vol. 104, no. 5, September 1999, discloses a new class of HIV inhibitors that block virus entry into host cells, namely secreted phospholipases A₂. In Ojwang et al., Antimicrobial Agents and Chemotherapy, vol. 39, no. 11, November 1995, there is described T30177, a potent inhibitor of laboratory strains and clinical isolates of HIV-1. Nakashima et al., Antimicrobial Agents and Chemotherapy, vol. 36, no. 6, June 1992, discloses the anti-HIV activity of a novel synthetic peptide, T-22. Its mechanism of action, possibly inhibition of virus-cell fusion, was found by a time-of-addition assay. Pannecouque et al., Current Biology, Vol. 12, Jul. 23, 2002 provides a new class of HIV integrase inhibitors that block viral replication in cell culture. Time-of-addition experiments indicated the mode of action of this class of inhibitors. In Pin-Fang et al., PNAS, Vol. 100, no. 19, Sep. 16, 2003, there is disclosed a small molecule HIV-1 inhibitor that targets the HIV-1 envelope and inhibits CD4 receptor binding. Time-of-addition studies were applied. In Pauwels et al., Nature, Vol. 343, Feb. 1, 1990, there is described potent and selective inhibition of HIV-1 replication in vitro by a novel series of TIBO derivatives. Spenlehauer et al., Virology, Vol. 280, No. 2, Feb. 15, 2001, provides a luciferase-reporter gene-expressing T-cell line which facilitates neutralization and drug-sensitivity assays that use either R5 or X4 strains of HIV-1. Richman et al., Journal of Virological Methods, Vol. 99, No. 1-2, January 2002, procies an adenovirus-based fluorescent reporter vector useful to Identify and Isolate HIV-infected cells. Lee A H et al., Biochemical and Biophysical Research Communications, Vol. 233, No. 1, Apr. 7, 1997, teaches the generation of the replication-competent HIV-1 which expresses a jellyfish green fluorescent protein. EP1335019 by Takeda Chemical Industries, Ltd. provides a T cell line carrying a reporter gene that contains an LTR sequence of HIV and expressing CCR5. Said T cell line is suitable for use in an efficient screening method for efficiently finding out a medicine such as an anti-HIV agent.

An internet article, February 2003, discloses a Staccato Cell Station™—Assay, by Zymark's Applied Science and Technology Group, which is an automation platform for processing a variety of cell based assays. In another internet article, April 2003, Hudson control group discusses the automation of cell-based assays and the application of robotics-based laboratory automation thereon.

In the search for HIV inhibiting compounds, time of addition (TOA) assays are valuable tools for identifying the mechanism of action of test compounds in relation to HIV viral entry, replication, integration and protease activity. Such assays generally comprise the infection of cells with HIV, and the addition of test compounds at various times following infection. Such assays are based on the principle that compound addition at or before the stage in the replication cycle inhibited by the compound results in ending of viral replication in that stage, while the same compound added after the stage in the replication cycle has no effect.

However, currently applied time of addition assays have the major disadvantage of being time-consuming and cost-effective. Also, such assays do not sufficiently and accurately permit the distinction between compounds that interfere with particular cellular events within a certain replication stage e.g. an entry, reverse transcription, integration stage, budding and maturation.

In view of the above, there is clearly a great need in the art for assays which overcome at least some of the above-mentioned disadvantages. It is therefore an object of the present invention to provide an improved assay for identifying novel drugs acting on particular stages in the HIV replication cycle. It is also an object of the present invention to provide novel anti-retroviral drugs. Furthermore, the invention aims to provide an apparatus for carrying out an improved assay according to the invention.

### SUMMARY

One embodiment of the present invention is a multi-well assay for identifying a compound Inhibiting the replication cycle of a micro-organism comprising the subsequent steps of:
a) preparing a multi-well comprising micro-organism-coated host cells,
b) initiating at time t micro-organism infection and replication in said micro-organism-coated host cells such that micro-organism infection and replication is initiated synchronically in all host cells,
c) bringing at time t+Δt a candidate compound at one or more concentrations into contact with a part of the host cells,
d) repeating step c) after a time interval of Δt for another part of said host cells,
e) optionally repeating steps c) and d) using one or more other candidate compounds at one or more concentrations, and
f) determining whether said candidate compound has inhibited micro-organism replication in said host cells.

Another embodiment of the present invention is an assay as described above wherein said micro-organism is HIV.

Another embodiment of the present invention is an assay as described above, whereby Δt is shorter than the time required for passing from one stage to another stage in the micro-organism replication cycle.

Another embodiment of the present invention is an assay as described above, whereby Δt is shorter than the time required for passing from the entry stage to the reverse transcription stage in the micro-organism replication cycle.

Another embodiment of the present invention is an assay as described above, wherein Δt, at which compounds are repeatedly added to the multi-well, comprises 1, 2, 3, 4, 5, 10, 15, 20, 25, 30, 40, 60, 120, 240 or 360 minutes.

Another embodiment of the present invention is an assay as described above, whereby steps c) to e) are performed under constant reaction conditions (including under a CO₂ concentration of 5%, a relative humidity comprised between 95 and 100% and a temperature of 37° C.).

Another embodiment of the present invention is an assay as described above, whereby micro-organism replication in said host cells is initiated at time t by simultaneously bringing all cells at a temperature suitable for initiating micro-organism replication.

Another embodiment of the present invention is an assay as described above, whereby said multi-well comprising micro-organism-coated host cells is prepared by the steps of
a) coating host cells with a micro-organism at a high multiplicity of infection,
b) removing unadsorbed micro-organism, and
c) bringing said micro-organism-coated host cells onto said well.

Another embodiment of the present invention is an assay as described above, whereby said host cells in said multi-well are able to express a gene encoding a detectable protein.

Another embodiment of the present invention is an assay as described above, wherein a vector that expresses a gene encoding a detectable protein under the control of a HIV responsive promoter is introduced in said host cells.

Another embodiment of the present invention is an assay as described above, wherein said micro-organism is labeled with a detectable protein reporting on infection.

Another embodiment of the present invention is an assay as described above, whereby determination of said candidate compound is performed by detecting the presence or absence of said detectable reporter.

Another embodiment of the present invention is an assay as described above, wherein the presence or the absence of said detectable protein is detected by means of digital imaging techniques.

Another embodiment of the present Invention is an apparatus for carrying out an assay as described above, comprising:
a support suitable for supporting a multi-well comprising micro-organism—coated host cells, optionally moving in one or more directions
one or more vials for containing a suspension of micro-organism,
one or more vials for containing one or more compounds,
pipetting means for dispensing micro-organisms in said multi-well, optionally moving in one or more directions
pipetting means for dispensing one or more compounds in said multi-well, optionally moving in one or more directions
pipetting controlling means for controlling dispensing by said pipetting means, and
environment controlling means for keeping conditions in said apparatus constant while bringing one or more compounds into contact with the host cells.

Another embodiment of the present invention is an apparatus as described above wherein said micro-organism is HIV.

Another embodiment of the present invention is an apparatus as described above further comprising a temperature-controlled multi-well support.

Another embodiment of the present invention is an apparatus as described above further comprising an insulating cover.

Another embodiment of the present invention is an apparatus as described above wherein the circuitry of pipetting and environment controlling means are sealed against high humidity.

Another embodiment of the present invention are compounds identifiable with an assay as described above.

Another embodiment of the present invention is a pharmaceutical composition comprising a therapeutically effective amount of one or more compounds identifiable with an assay as described above and a pharmaceutically acceptable excipient.

Another embodiment of the present invention is a use of a compound, identifiable with an assay as described above, as a medicament.

Another embodiment of the present invention is a use of a compound, identifiable with an assay as described above, for the manufacture of a medicament for treating infectious diseases.

Another embodiment of the present invention is a method of treating AIDS, comprising administering a therapeutically effective amount of a compound identifiable with an assay as described above to a patient In need thereof.

According to the present Invention a safe, cost-effective multi-well assay was developed, which enables the analysis of hundreds of thousands of compounds for anti-retroviral activity in an automated and rapid way. In particular, the present assay allows the screening of compounds for antiretroviral activity during the entire HIV-1 life cycle.

Therefore, in a first embodiment the invention provides a multi-well assay for identifying a compound inhibiting the replication cycle of a micro-organism comprising the subsequent steps of:
a) preparing a multi-well comprising micro-organism-coated host cells,
b) initiating at time t, micro-organism infection and replication in said host cells such that micro-organism infection and replication is initiated synchronically in all host cells,
c) bringing at time t+Δt a candidate compound at one or more concentrations into contact with a part of the host cells,
d) repeating step c) after a time interval of Δt for another part of said host cells,
e) optionally repeating steps c) and d) using one or more other candidate compounds at one or more concentrations, and
f) determining whether said candidate compound has inhibited micro-organism replication in said host cells.

A micro-organism according to the invention may be any of interest that has a replication cycle. Examples include Staphylococcus aureus, Mycobacterium tuberculosis, Respiratory Syncitial Virus, Hepatitis B virus (HBV), Hepatitis C virus (HCV), Herpes simplex virus, and preferably Human immunodeficiency virus (HIV).

A first main step in the assay according to the invention comprises the preparation of a multi-well comprising micro-organism coated host cells. The main particularity of this step is that the well is prepared in such a way that micro-organism replication is simultaneously initiated in all micro-organism-coated host cells on the multi-well. This may in particular be obtained by removing unadsorbed micro-organism after interaction of micro-organism with the host cells and prior to transferring the micro-organism infected host cells to a multi-well plate. In addition, synchronization of micro-organism replication initiation can be obtained by bringing at a same time t, micro-organism coated host cells on the multi-well at a temperature suitable for initiating micro-organism replication.

Secondly, test compounds are brought into contact with the micro-organism coated host cells onto the multi-well at different times. The main particularity of this second step is that compounds are added to the multi-well after a time interval Δt, which is shorter than the time required for passing from one stage to another stage in the micro-organism replication cycle.

The present assay allows screening of compounds having an antiretroviral activity at various stages of micro-organism replication, e.g. at the entry stage, the reverse transcription stage, the integrase stage and the protease activity stage. Thus, the present assay advantageously permits screening of compounds for antiretroviral activity during the entire micro-organism replication cycle.

In addition, as the time interval for adding test compounds to the multi-well are shorter than the time for completing a stage in the replication cycle, the assay permits to screen compounds that interfere with particular events within a certain micro-organism replication stage.

Furthermore, because micro-organism replication is initiated at the same time in all micro-organism coated host cells on the well, inter-well variation in micro-organism replication is substantially reduced and the assay permits screening of compounds for activity at relatively high speed by adding test compounds after the onset of micro-organism replication. The assay thus allows the identification of compounds having an activity at very early stages of micro-organism replication, for instance already during the entry stage of micro-organism replication.

In a particularly preferred embodiment, Δt is shorter than the time required for passing from the entry stage to the reverse transcription stage in the micro-organism replication cycle. By choosing time intervals Δt which are shorter than the entry stage of micro-organism in a host cell, the assay enables the identification of compounds that act on particular cell-virus interaction events that take place during the entry stage of micro-organism replication.

A third main step in the assay comprises rapid and sensitive detection of inhibition of micro-organism replication by tested compounds. As such, the micro-organism-infected cells in the multi-well are able to express a gene encoding a reporter protein. Alternatively, micro-organisms may also be labeled with a detectable. Inhibition by a tested compound is determined by detecting the presence or absence of said reporter.

Using the assay according to the present invention thus permits the identification of novel drugs acting on particular stages in the micro-organism replication cycle and also to identify into more detail the specific mode of action of anti-micro-organism drugs within a certain stage in the replication cycle, for instance within the entry stage.

In another embodiment, the present invention relates to an apparatus for carrying out an assay according to the present invention.

The invention further relates to compounds identifiable with an assay according to the invention and pharmaceutical composition comprising a therapeutically effective amount of such compounds. The identified compounds may be used as medicaments, and in particular for the manufacture of a medicament for treating micro-organism infections and in methods for treating micro-organism infections.

Other objects and advantages of the present invention will become apparent from the following detailed description taken in conjunction with the accompanying figures and examples.

### FIGURES

FIG. 1 represents a perspective view of an apparatus according to the present invention.

FIG. 2 represents a topical view of an apparatus according to the present invention.

FIG. 3 represents the results of an assay according to the classical TOA protocol wherein the effect of reference compounds having known activity on HIV-infected cells are tested at different time points after addition to the HIV-coated cells in a 96-well plate.

FIG. 4 represents the results of an assay according to the invention wherein the effect of reference compounds having known activity on HIV-infected cells are tested at different concentrations.

FIG. 5 represents the results of an assay according to the invention wherein cell toxicity effects of reference compounds having known activity on HIV-infected cells are tested at different concentrations.

FIG. 6 represents the results of an assay according to the invention wherein the effect of reference compounds having known activity on HIV-infected cells are tested at different time points after addition to the HIV-infected cells in a 96-well plate.

FIG. 7 represents the results of an assay according to the invention wherein the effect of reference compounds having known activity on HIV-Infected cells are tested at different time points after addition to the HIV-infected cells in a 384-well plate.

FIG. 8 represents an Image of an assay according to the invention wherein the effect of reference compounds having known activity on HIV-infected cells are tested at different time points, in duplicate, after addition to the HIV-coated cells In a 384-well plate.

FIGS. 9a and 9b represent the results of an assay of the Invention, wherein the effects of the inhibitor BMS806 upon the activity of HIV-infected cells is evident when the incubation temperatures are 23 deg C. (9a) and 37 deg C. (9b). The inhibitor is added at regular intervals, starting at time delta t (30 mins, 1, 2, 4, 6, and 8) as indicated in the legend. In 9a cells are incubated at 23 deg C. for 4 hours after which the replication cycle was allowed to proceed at 37 deg C. until read out. In 9b the cells were incubated at 37 deg C. during the entire experiment.

FIGS. 10a and 10b represent the results of an assay of the invention, wherein the effects of the inhibitor AMD3100 upon the activity of HIV-infected cells is evident when the incubation temperatures are 23 deg C. (10a) and 37 deg C. (10b). The inhibitor is added at regular intervals, starting at time delta t (30 mins, 1, 2, 4, 6, and 8) as indicated in the legend. In 10a cells are incubated at 23 deg C. for 4 hours after which the replication cycle was allowed to proceed at 37 deg C. until read out. In 10b the cells were incubated at 37 deg C. during the entire experiment.

FIGS. 11a and 11b represent the results of an assay of the invention, wherein the effects of the inhibitor T-20 upon the activity of HIV-infected cells is evident when the incubation temperatures are 23 deg C. (11a) and 37 deg C. (11b). The inhibitor is added at regular intervals, starting at time delta t (30 mins, 1, 2, 4, 6, and 8) as indicated in the legend. In 11a cells are incubated at 23 deg C. for 4 hours after which the replication cycle was allowed to proceed at 37 deg C. until read out. In 11 b the cells were incubated at 37 deg C. during the entire experiment.

### DETAILED DESCRIPTION OF THE INVENTION

Current HIV therapies involve the use of inhibitors of reverse transcriptase (RT), viral entry and protease enzymes. Despite the development of novel classes of inhibitors and complex drug regimens, drug resistance is increasing. Thus, new types of anti-HIV drugs are continuously necessary.

Assay

The present invention relates in an embodiment to a multi-well assay for identifying novel compounds which inhibit the HIV replication cycle. The assay provides a convenient model for high-throughput screenings of compounds that show anti-retroviral activity and that can interfere with HIV replication in living cells. Briefly, the present assay comprises three main steps. First, a multi-well comprising HIV-coated host cells is prepared. Secondly, test compounds are brought into contact with HIV-coated host cells applied on the multi-well at different times. A large number of different compounds, optionally at different concentrations, can be applied on the multi-well for testing antiretroviral activities of these compounds. A third step in the assay comprises rapid and sensitive detection of inhibition of HIV replication by the tested compounds.

In particular, the assay comprises the subsequent steps of:
a) preparing a multi-well comprising HIV-coated host cells under conditions suppressing HIV replication,
b) initiating at time t HIV infection and replication in said HIV-coated host cells such that HIV replication is initiated synchronically in all host cells,
c) bringing at time t+Δt a candidate compound at one or more concentrations into contact with a part of the HIV-coated host cells,
d) repeating step c) after a time interval of Δt for another part of said HIV-coated host cells,
e) optionally repeating steps c) and d) using one or more other candidate compounds at one or more concentrations, and
f) determining whether said candidate compound has inhibited HIV replication in said HIV-infected host cells.

As used herein “human immunodeficiency virus (HIV)” refers to any HIV strain including laboratory HIV strains, wild type HIV strains, mutant HIV strains and any biological sample comprising at least one HIV virus, such as, for example, a HIV clinical isolate. HIV strains compatible with the present invention are any such strains that are capable of infecting mammals, particularly humans. Examples are HIV-1 and HIV-2.

“HIV replication cycle” or “HIV live cycle” or similar terms as used herein refers to the complete cycle passed through by HIV during its replication, starting with contact and attachment of HIV to a host cell and ending with the production of new viral particles. The HIV replication cycle is meant to encompass various stages, which can be broadly identified as a) an entry stage, b) a reverse transcription stage, c) an integration stage, d) a budding stage and e) maturation stage (in which protease plays an important role).

“HIV-coated host cells” as used herein refers to host cells that have been brought into contact with HIV, but wherein replication of HIV has not yet started. Host cells are meant to encompass eukaryotic cells, and preferably mammalian cells from human or animals. In principle, all cells which are susceptible to HIV and which HIV can infect may be used in accordance with the present invention. Preferred host cells for use in assays of the present invention comprise but are not limited to animal or human cells such as CHO, COS, HeLa, C127, 3T3, BHK, HEK 293, Bowes melanoma cells, MT4, D17, etc.

“HIV-infected host cells” as used herein refers to the host cells referred above that have been brought into contact with HIV, and wherein replication of HIV has started. Replication starts when the host cells are brought into contact with HIV at an incubation temperature of 37° C.

Although the embodiments described in this section relate to HIV, the method and device of the invention may be applied by the skilled person towards any micro-organism of interest that has different life cycle stages. Examples include Staphylococcus aureus, Mycobacterium tuberculosis, Respiratory Syncitial Virus, Hepatitis B virus (HBV), Hepatitis C virus (HCV), Herpes simplex virus, and preferably Human immunodeficiency virus (HIV).

The assay according to the invention is performed in a multi-well format, which is suitable for screening large amounts of compounds for anti-retroviral activity in an automated and rapid way. The multi-well plates used in the present invention are intended to encompass all sizes of multi-well plates, and preferably comprise 96-well or 384-well plates.

The multi-well plates according to the present invention are prepared by coating host cells with HIV at a high multiplicity of infection, removing unadsorbed HIV, and bringing said HIV-coated cells onto said well.

By bringing the cells into contact with HIV-1, the infection can be initiated by incubation at 37° C. Preferably the multiplicity of infection (MOI) is higher than 1.0, and more preferably higher.

Removing unadsorbed HIV after interaction of HIV with the host cells permits the removal of residual HIV that has not interacted with the host cell and which could disturb the assay and provides unreliable results.

The coated host cells are then transferred to a multi-well plate. Preferably the concentration of HIV-coated host cells per well is preferably between 200,000 and 600,000 cells per well, and even more preferred comprises 500,000 cells per well.

As used herein, “multiplicity of infection” (MOI) refers to the ratio of the number of virus to the number of cells when a virus is used to contact cells. It is also being referred to as the virus infectivity titer, and is the average number of infectious viruses per cell when cells are infected with the virus. It also represents the number of colony-forming units added per cell, and its units are expressed as CFU/cell. For example, a MOI of 1 represents one infectious retroviral particle per target cell. Multiplicity of infection (MOI) is calculated from cell number as described in Martuza et al. Science 252:854 (1991), or by alternative methods known by the skilled in the art. Petri dishes, and multi-well plates are commonly used.

In a preferred embodiment, once the HIV-coated host cells have been transferred to a multi-well plate, HIV replication is preferably initiated in all HIV coated host cells at the same time t by bringing all host cells on the multi-well at a temperature suitable for initiating HIV replication, being preferably 37° C.

In a next step, test compounds are added to the multi-well plate comprising the HIV infected cells at various times following Initiation of HIV replication. In principle, compound addition at or before a stage in HIV replication, which is inhibited by the compound, results in reduction of virus growth, while compound addition after a step in HIV replication, which is inhibited by the compound, has no effect.

As used herein “compounds” may refer to known drugs having antiretroviral activity as well as to compounds having unknown antiretroviral activity. The assay according to the invention permits to identify novel compounds having antiretroviral activity as well as to identify Into more detail the specific mode of action of readily known anti-HIV drugs during/within a certain stage in the replication cycle.

It will be clear from the present invention that several compounds may be tested simultaneously at one or different concentrations. Time t according to the invention is the time at which HIV replication is initiated in all or most of the cells in the multi-well. After a time t+Δt, a compound(s) is transferred to a part of the wells in the multi-well plate. After various additional intervals Δt, the same compound(s) may be transferred to other, not yet treated, wells of the multi-well plate, in order to screen the compound(s) for potential HIV replication inhibiting effects in function of their addition time to the infected HIV cells. This allows evaluating at which particular stage in the HIV cycle the compound(s) interfere with HIV replication.

In addition, since the time intervals Δt is selected in order to be shorter than the time required for passing from one stage to another stage in the HIV replication cycle, the assay allows evaluating at which particular stage in the HIV cycle the compounds Interfere with HIV replication.

A candidate compound is preferably brought into contact with a part of the HIV-infected host cells in said multi-well at a time t+Δt, whereby Δt is shorter than the time required for passing from one stage to another stage in the HIV replication cycle. According to this embodiment, the present invention permits to screen compounds for antiretroviral activity within various stages of the HIV replication cycle, e.g. within the entry stage, the reverse transcription stage, the integrase stage or the protease activity stage. Furthermore, because HIV replication is initiated at the same time in all HIV-coated host cells on the well, the assay permits to test the effect of compounds on HIV replication at relatively high speed by adding compounds after the onset of HIV replication. The assay thus allows identifying compounds having an antiretroviral activity at very early stages of HIV replication, e.g. compounds that interfere with the entry stage of HIV replication, which is generally performed within 30 minutes post replication Initiation. Thus, the present assay advantageously permits screening of compounds for antiretroviral activity at early as well as at late stages during the entire HIV life cycle.

In a particularly preferred embodiment, Δt is shorter than the time required for passing from the entry stage to the reverse transcription stage in the HIV replication cycle. This allows screening for compounds interfering with events taking place during particular phases within the entry stage of HIV replication. Briefly, the entry stage of HIV replication distinguishes a CD4 receptor attachment phase, a co-receptor binding phase and a phase involving membrane fusion events. By choosing time intervals Δt, which are shorter than the entry stage of HIV in a host cell, the assay enables the identification of compounds that have anti-retroviral activity during one or more of these above-referred phases of the entry stage. In particular, the present invention so allows the identification of compounds interfering with either the CD4 receptor attachment phase, the co-receptor binding phase and/or the membrane fusion phase. It is clear that compounds having antiretroviral activity at very early stages in HIV replication, in particular even at particular phases during the entry stage of HIV, are of particular importance for anti-HIV therapies, since rapid inhibition of HIV replication increases the chances of successfully treating HIV.

In a preferred embodiment, the time intervals Δt, at which compounds are repeatedly added to the multi-well, comprises 10 sec, 20 secs, 40 secs, 50 secs, 1 min, 2, 3, 4, 5, 10, 15, 20, 25, 30, 40, 60, 120, 240 or 360 minutes.

In another preferred embodiment, reaction conditions applied during the addition of compounds to the multi-well in steps c) to e) of the assay are kept constant.

In one embodiment of the invention, the temperature T of the wells is determined by the user. For example, a temperature of 5, 6, 7, 8, 9, 10, 11, 12, 13, 14, 15, 16, 17, 18, 19, 20, 22, 23, 24, 25, 26, 27, 28, 29, 30, 31, 32, 33, 34, 35, 36, 37, 38, 39, 40, 41, 42, 43, 44, 45, 46, 47, 48, 49, 50 or higher might be selected, and is kept constant throughout out the experiment.

In one embodiment of the invention, the temperature of every well in the multiwell is the same.

In one embodiment of the invention, the temperature of at least one well is different from another. For example, a temperature gradient might be held across the width of the multi-well plate such that the left side of the plate is hotter than the right side of the plate. Alternatively, the temperature of an individual well in a multiwell plate may be different.

In another embodiment of the invention, a means is present to prevent the contents of the wells from reducing in volume by evaporation. For example, a relative humidity of between 95 and 100% is provided. Alternatively, a removable heated lid is provided which is applied on top of the multiwell plate between additions of reagents.

Preferably, the following conditions are maintained: a CO₂ concentration of 5%, a relative humidity comprised between 95 and 100% and a temperature of 37° C.

Inhibition of HIV replication by a compound is determined by detecting the presence or absence of a detectable protein, which is released either by the host cell or by the virus as such.

In one embodiment, HIV-infected cells in the multi-well are engineered in order to be capable of expressing a gene encoding a detectable (reporter) protein. Preferably, a gene encoding a detectable protein is put under the control of a HIV responsive promoter and introduced in said host cells. The reporter construct can be transferred into the host cells, for example by transfection, microinjection etc. . . . HIV replication in the host cell triggers expression of the gene and presence of the detectable protein indicates successful HIV replication in the cell.

The term “HIV responsive promoters” as used In the present invention is meant to include any transcriptional control unit capable of initiating transcription upon initiation of virus replication and includes regulatory elements such as enhancers and other regions binding transcriptional control factors. Examples of suitable HIV responsive promoters are known in the art.

Suitable genes encoding reporter proteins according to the present invention may be any reporter gene known to the skilled person as being active in cells. The reporter gene preferably encodes an enzyme which catalyses a reaction or may be a protein which produces a detectable signal, preferably a visually detectable signal, such as a colored product. Examples of suitable reporter genes comprise but are not limited to genes encoding chloramphenicol acetyltransferase (CAT), β-galactosidase (GAL), β-glucuronidase (GUS), luciferase (LUC), green fluorescent protein (GFP), etc.

In another embodiment, HIV is labeled with a marker like a detectable protein or lipotracer. Examples methods of labeling an organism using a detectable protein are known in the art and include, for example, the use of lipotracers, VRP-GFP or any suitable GFP-fusion (Mc Donald et al, JCB 159, 3, pp441-452).

In one embodiment, the detectable protein is detected directly, in case of a colored, fluorescent or luminescent protein. Fluorescence, for example that is produced as a result of luciferase activity, may be quantitated using a spectrophotometer.

The presence and/or amount of gene product resulting from expression from the reporter gene may also be determined indirectly by using immunolabelling techniques.

The term “immunolabelling technique” as used herein is meant to refer to various detection methods that use immunoglobulins to detect specific epitopes. Such immunolabelling technique may comprise but are not limited to ELISA, immunostaining, immunohistochemistry, enzyme immunoassays, Western blotting, flow cytometry, nephelometry, immunosensors.

Detection is preferably performed by means of digital imaging techniques. Such techniques are known in the art and include, for example, the use of a luminometer, scanning microscope, spectrophotometer, and preferably a fluorescence reader.

The present invention is particularly advantageous in that the effect of different antivirals on each of the three stages involved during the entry process of the HIV replication cycle, i.e. CD4 receptor attachment phase, co-receptor binding phase, and membrane fusion events, are accurately and precisely visualized. The invention provides an assay which determines upon which specific stages within the HIV replication cycle that a compounds acts. This precise and accurate identification and visualization of the mode of action of an antiviral is partly due to the automation of the time-of-addition assays herein presented. Because of this automation, the assay may be monitored at shorter intervals of times, i.e. at each minute, or even within a seconds range.

Apparatus

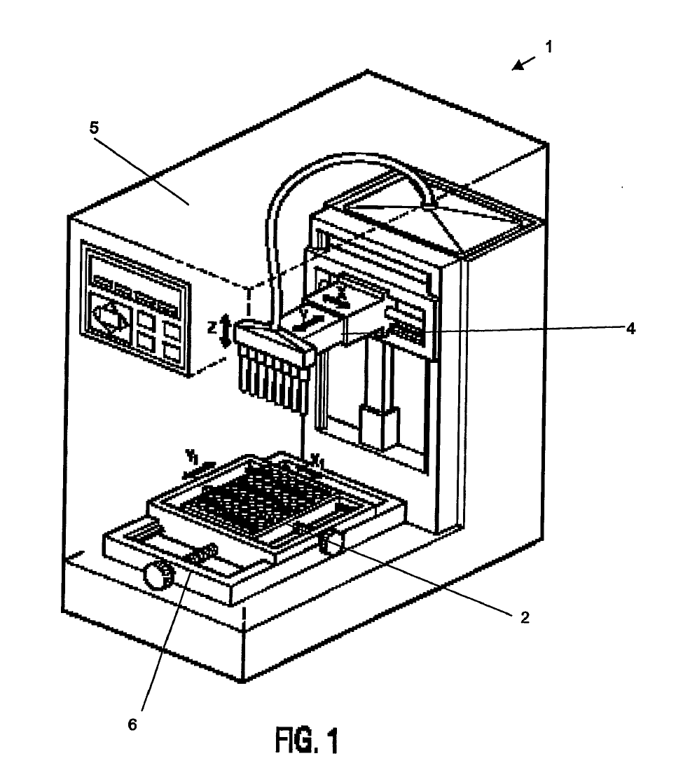

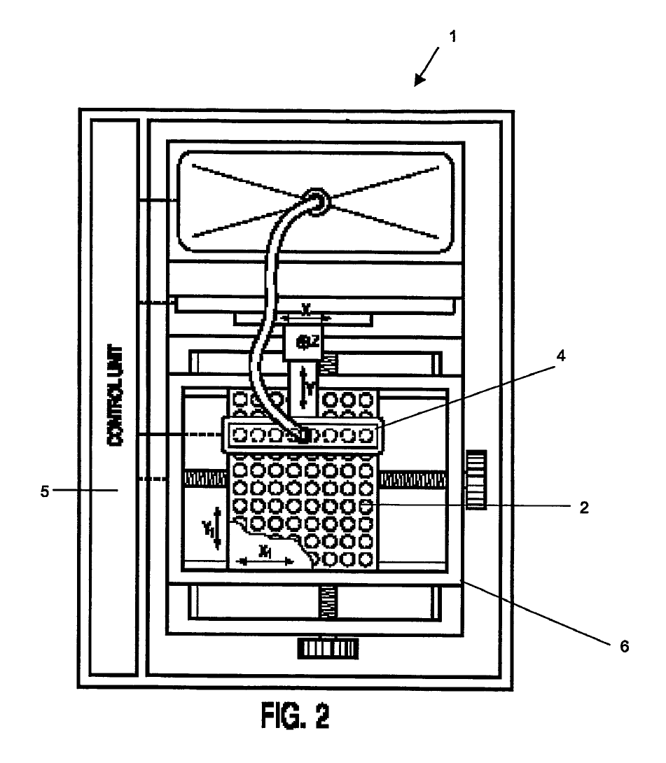

In another embodiment, the invention relates to an apparatus for carrying out an assay according to the present invention. The apparatus 1, of which an example is illustrated in FIG. 1 and FIG. 2, comprises
a support suitable for moving in one or two directions for supporting a multi-well comprising HIV-coated host cells,
one or more vials or wells of a microtiter plate for containing one or more compounds,
pipetting means for dispensing one or more compounds in a multi-well, positionable on said support,
controlling means for controlling dispensing by said pipetting means,
controlling means for controlling the reaction conditions in said apparatus,
controlling means for temperature,
optionally controlling means for humidity.

As illustrated in FIG. 1 and FIG. 2 an apparatus is represented comprising a support 2 whereon a multi-well comprising HIV-infected host cells can be positioned. The support is preferably movable, in one or two directions, e.g. in X1 and Y1 direction. Therefore, the support 2 can for instance be positioned on a X-Y table 6. Such X-Y tables are well known in the art and are therefore not described into detail in the present specification.

Alternatively, the support 2 may be stationary, and an arm for liquid dispensing 4 is movable in one or more directions e.g. in Y1 and optionally in X1.

Alternatively, both an arm as described above and an X-Y table may be movable according to a device of the invention.

In another embodiment of the invention, the plate support is heated so as to maintain the temperature of the multiwell plate according to the method. It is an aspect of the invention, that the plate support has the means to maintain the same and constant temperature across its surface. It is another aspect of the invention, that the plate support has the means to maintain a constant temperature gradient across its surface (e.g. a temperature gradient from left to right). It is another aspect of the invention, that the plate support has the means to maintain a constant and different temperature in each well of the multiwell plate.

In another embodiment of the present invention, the control of the temperature is optimized such that the entire area is kept constant in temperature. If necessary, the temperature can be incrementally increased or decreased. Examples of temperatures are 4° C., room temperature (±22° C.) or 37° C.

In a preferred embodiment, the apparatus comprises one or more containers holding the compounds to be tested. The container may be one or more multi-well plates, vials or container known in the art. The transfer of the compounds to the multi-well plate, which is to be positioned on the support 2, can be performed by pipetting means 4. Preferably, these pipetting means 4 are movable in three directions, X, Y and Z.

The present apparatus further comprises one or more controlling means. A control means 5 is provided for controlling the reaction conditions in the apparatus, before as well as after initiation of HIV replication. At the beginning of the assay, the conditions of the apparatus should be regulated such that HIV replication is suppressed. Only at time t, HIV replication should be initiated for all cells in the multi-well at the same time by controlling the temperature in the apparatus. In addition, the reaction conditions in the incubator should be kept constant after initiation of replication and during an after addition of compounds to be tested, such that all compounds can be screened under similar reaction conditions. Therefore, the controlling means monitors and maintains parameters such as plate temperature, ambient air temperature, ambient air humidity, and the gaseous composition of the ambient air.

It is an aspect of the invention that the apparatus is enclosed in an environment in which the ambient air is controlled, for example the level of CO₂, the temperature and/or humidity may be controlled by the user. It is an aspect of the invention that the electronics are present In the same environment as the multiwell plate and the electronics are sealed so as to prevent the humidity, temperature and CO₂ from Interfering with their normal function.

It is an aspect of the invention that the environment is enclosed by cover means that seal the device substantially from the environment of the user. Said cover is part of the device. The cover may be partially or totally transparent to enable the user to see the multiwell plate. The cover may be have a insulative properties enable so as to stabilize the temperature. The cover may be airtight, so as to stabilize the level of CO₂.

Further control means are preferably provided for controlling the pipetting action of the pipetting means 4. This control means may be similar or different from the control means 5 for regulating the reaction conditions in the apparatus. The pipetting control means are suitable for determining and regulating the intervals Δt at which compounds need to be added to the multi-well.

It is an aspect of the invention that the control means, and the pipettor controlled thereby are capable of dispensing nanolitre volumes. Examples of automatic dispensing devices may be any of the art. They include those capable of delivering down to nonalitre volumes and may be operated by micro pumps, and piezo pumps.

In another embodiment of the invention, the apparatus is further provided with a heated lid which is brought into contact with the top surface of the multiwell plate during incubations. The temperature of the well-side of the lid is maintained sufficiently high to prevent the contents of the well from evaporating. The lid may have a means to seal with the surface of the multiwell plate, such as a gasket. The lid is mechanically brought into contact with the top surface of the multiwell plate during incubations. During an addition operation, the lid is mechanically opened. The lid may be mechanically opened by any mechanised means, such as, for example, a robot arm, a motorized hinge etc.

The apparatus according to the invention has the advantage of enabling compound testing at various time intervals Δt, without the need of taking the multi-well plate out of the apparatus every time compounds need to be added to the multi-well. This can be obtained by providing all necessary equipment for screening compounds for antiretroviral activity within apparatus 1, and by providing means 5 which permit to control and set up constant reaction conditions in the apparatus.

In another preferred embodiment, the apparatus is further provided with detecting means for determining whether the tested compounds have inhibited HIV replication. These detecting means may for instance comprise imaging means.

In yet another preferred embodiment, the apparatus is further provided with analyzing means for analyzing the effects of tested compounds on HIV replication. These analyzing means may for instance comprise computer means, suitable for registering and determining at which stage replication, or at which phase within a replication stage the compounds show antiretroviral effects.

In another preferred embodiment, the apparatus according to the invention preferably comprises different units, including a loading unit, wherein test compounds are transferred to the multi-well plates, a detection unit, wherein potential inhibitory effects of tested compounds are determined, and an analysis unit, wherein the effects of the test compounds are analyzed. Preferably, the apparatus comprises transport means, for transporting the multi-well plates from one unit to another in said apparatus.

Medicinal Uses

It was found that the present assay provides the necessary sensitivity for identifying antiviral molecules. Thus, the assay according to the invention can be utilized to facilitate the development of a safe, efficient method for screening compound libraries for anti-HIV activity.

Reach Through Products

In a further aspect the invention relates to compounds identifiable with an assay according to the invention. The compounds are particularly suitable for being applied in AIDS therapies. The term “AIDS (acquired immunodeficiency syndrome)” refers to the late stage of HIV disease, characterized by a deterioration of the immune system and a susceptibility to a range of opportunistic infections and cancers.

In another further embodiment, a pharmaceutical composition is provided comprising a therapeutically effective amount of one or more compounds identifiable with an assay according to the invention and a pharmaceutically acceptable excipient.

The invention also relates to the use of a compound, identifiable with an assay according to the invention as a medicament or for the manufacture of a medicament for treating AIDS.

In yet another embodiment, the invention relates to a method of treating AIDS, comprising administering a therapeutically effective amount of a compound identifiable with an assay according to the invention to a patient in need thereof.

The following examples are meant to illustrate the present invention. These examples are presented to exemplify the invention and are not to be considered as limiting the scope of the invention.

### EXAMPLES

### Example 1

In a time-of-addition experiment according to the present invention, the step in the HIV replication cycle in which a compound was active was determined and compared with reference compounds including inhibitors for binding/fusion (entry process), reverse transcriptase, integrase and protease. When a potent antiviral compound was added at the time of infection, no viral replication took place. But, if addition of compound was delayed, protection against HIV replication could be observed up to the moment that the virus passed the stage at which the inhibitor interacts. The use of reference compounds with a known mode of action was essential for the correct interpretation of the results.

A MT4 cell line expressing EGFP reporter gene under the control of a HIV-1-LTR promoter element was used. MT4-LTR-EGFP cells were exposed to HIV at a high multiplicity of infection (MOI) by centrifugation for 10 min at 400 g. Unadsorbed virus was then removed by two washing steps at 4° C. in order to synchronize the infection. Multi-well plates loaded with cells were then incubated at 37° C., a temperature suitable for initiating the HIV replication cycle.

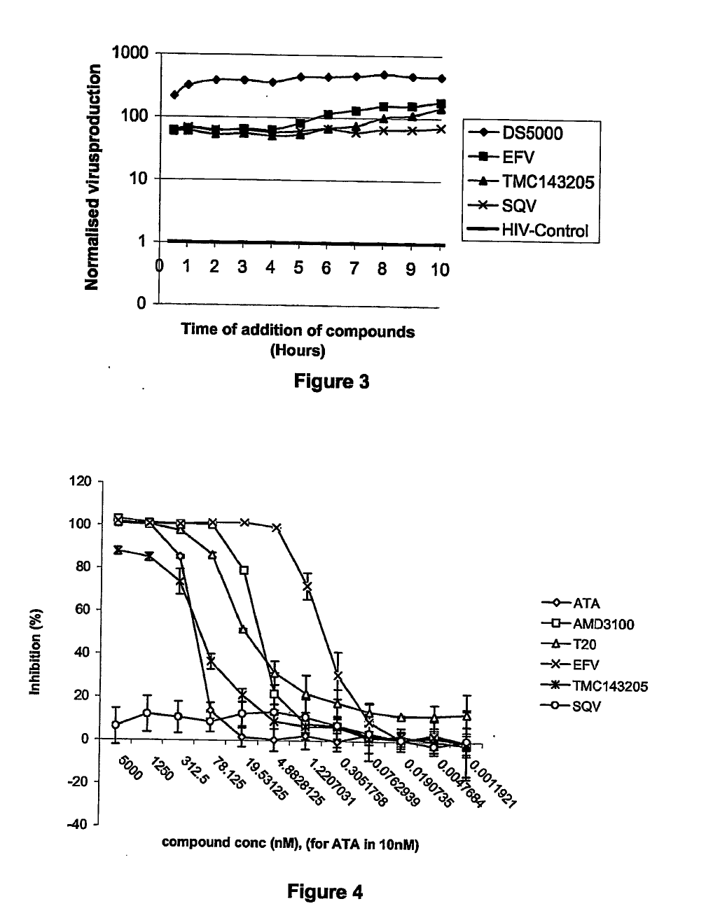

From 5, 10, 15, 20, 30 or more min post-infection on, compounds to be tested were added to parallel cultures in micro-titer plates at different times. The cultures were scored microscopically for fluorescence 24 hours after infection and supernatant was collected. HIV replication in the supernatant samples was quantified by measuring the concentration of the p24 viral antigen using a commercial kit, according to the manufacturer protocol (NEN). FIG. 3 shows that based on p24 production which is related to virus production entry (DS5000=dextrane sulfate), reverse transcription (EFV=efavirenz) and integration (TMC143205=integrase Inhibitor) can be distinguished.

### Example 2

The present example illustrates a method according to the invention for identifying compounds having an inhibiting effect on HIV during the entry process of the HIV replication cycle.

A MT4 cell line was used In the present example expressing a luciferase reporter gene under the control of a HIV-1-LTR promoter element. Cells were counted and a cell suspension of 500.000 ml or 350.000 ml was made for use in 96-well or 384 wells, respectively. The cells were centrifuged for 10 minutes at 400 g. Subsequently, cells were infected with at high multiplicity of infection with HIV-1 (strain 111B) at a host cell/virus titer of 1/1. Unabsorbed virus was removed by two washing steps in order to synchronize the infection. After the second washing step, cells were resuspended and the suspensions were distributed in the wells of a micro-titer plate in an amount of 95 μl or 36 μl in a 96-well or a 384 well format, respectively. At predefined time points 5 μl (96 well) and 4 μl (384 well) of compound was added. As compounds to be tested, reference compounds having well known mechanisms of action were included in the plates.

2.1 Titration of Compounds Under TOA-Conditions

96-well plates loaded with cells coated with viruses as explained above were incubated at 37° C., being a temperature suitable for initiating the HIV replication cycle. 30 minutes post-infection; a dilution series of compounds to be tested was added to the wells of the micro-titer plate in an amount of 5 μl/well. The plates were further incubated at 37° C. for 26 hours.

As compounds to be tested, reference compounds having well known mechanisms of action were included in the plates. The reference compounds comprised:
ATA, AMD 3100 and T20 having inhibiting effects on the HIV entry process,
EFV having inhibiting effects on the HIV reverse transcriptase process,
TMC143205 having inhibiting effects on the HIV integration process,
SQV having inhibiting effects on the HIV protease activity process.
ATA was used in a concentration from 5 μM to 3×10⁻⁶ μM; while all other compounds were used in a concentration from 5 μM to 3×10⁻⁷ μM.

Toxicity measurements, with the aim of determining compounds that demonstrate the most activity for the least toxicity were performed in parallel with activity testing of the cited compounds. Non-specific activity of the compounds was measured by incubating the same cells as described above using the same protocol as described above, without addition of virus particles.

In order to determine whether said test compounds had inhibited HIV replication in the host cells, the presence or absence of the luciferase gene product was measured.

Therefore, 100 μl Luc-lit™ substrate was added to each well and luminescence was monitored by processing the plates in a PerikinElmer-Viewlux™ within 30 minutes. HIV replication was quantified by measuring the luciferase signal.

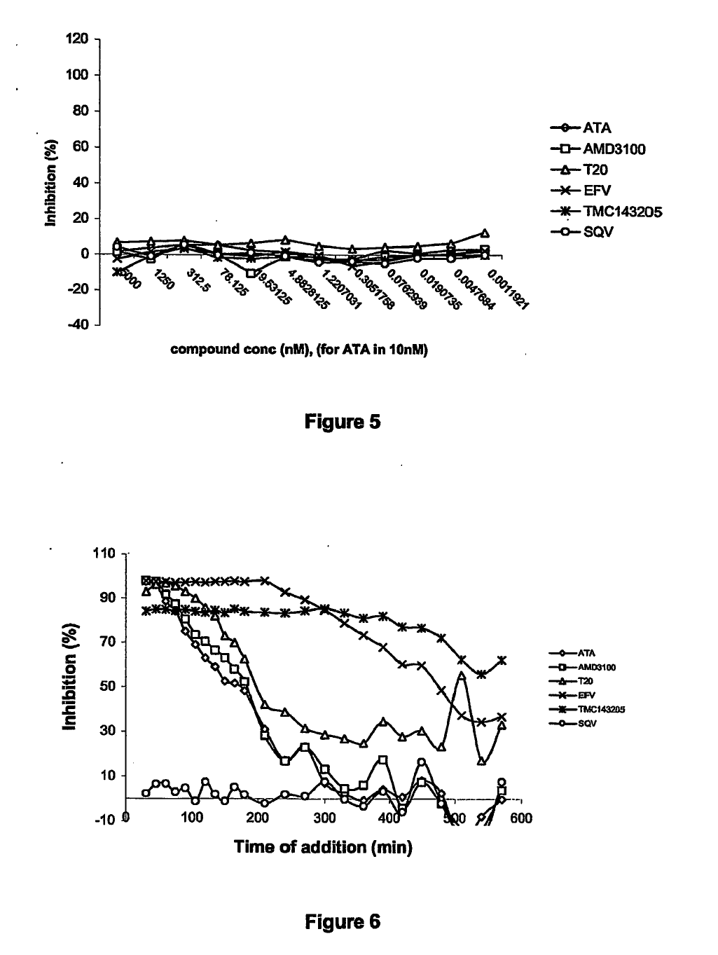

Dose response curves for activity and toxicity are presented in FIG. 4 and FIG. 5 respectively. From the figures it can be seen that the highest compound concentration used in the assays exhibited no toxicity (FIG. 5) and gave approximately 100% inhibition (FIG. 4). The results demonstrate that the method of the invention enable the selection of compounds which exhibit the most activity for the least toxicity.

2.2 Time Aspect of the Method According to the Invention Using Luminescent Cells

96-well plates prepared as indicated above were incubated at 37° C., a temperature suitable for initiating the HIV replication cycle. 30 minutes post-infection; a dilution series of compounds to be tested was added to the wells of the micro-titer plate in an amount of 5 μl/well at different time points. The plates were further incubated at 37° C. for 16 hours.

Also 384-well plates, prepared as indicated above, were incubated at 37° C., being a temperature suitable for initiating the HIV replication cycle. 30 minutes post infection; compounds to be tested were added to the wells of the micro-titer plate in an amount of 4 μl/well. The plates were further incubated at 37° C. for 16 hours.

The reference compounds as indicated in 1.1 were tested. ATA was used in a concentration of 5 μM; AMD3100, T20 EFV ad SQV were used in a concentration of 1 μM and TMC143205 was used in a concentration of 1 μM. In this example, the time points for adding the compounds comprised 30, 45, 60, 75, 90, 105, 120, 135, 150, 165, 180, 210, 240, 270, 300, 330, 360, 390, 420, 450, 480, 510, 540 and 570 minutes. 100 μl Luc-lit™ substrate was added to each well and luminescence was monitored by processing the 96- and 384-well plates in a PerkinElmer-Viewlux™ within 30 minutes. HIV replication was quantified by measuring the luciferase signal.

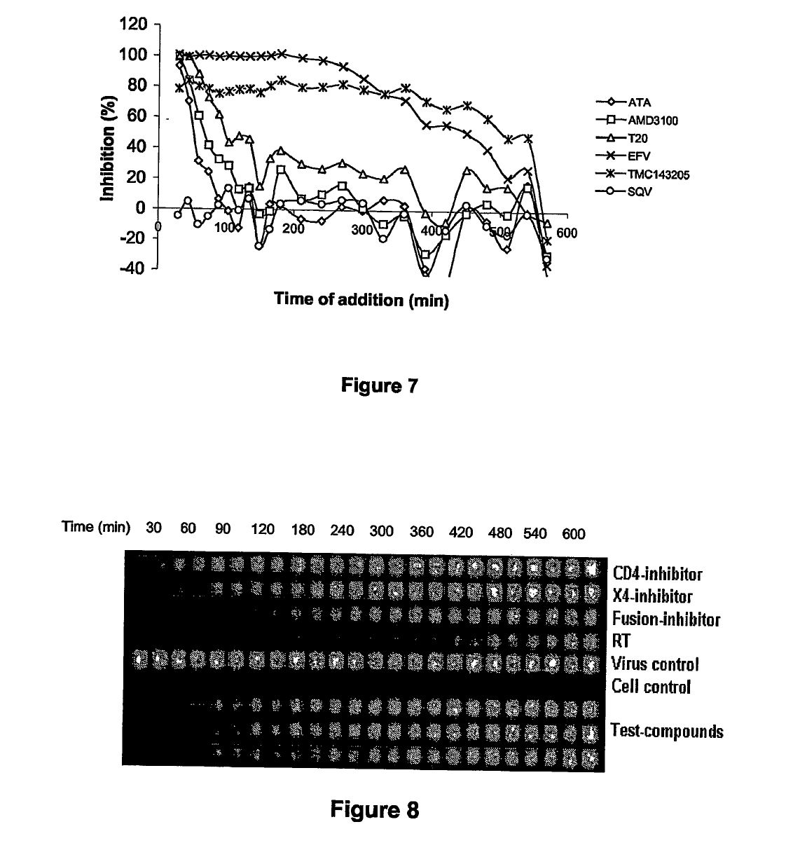

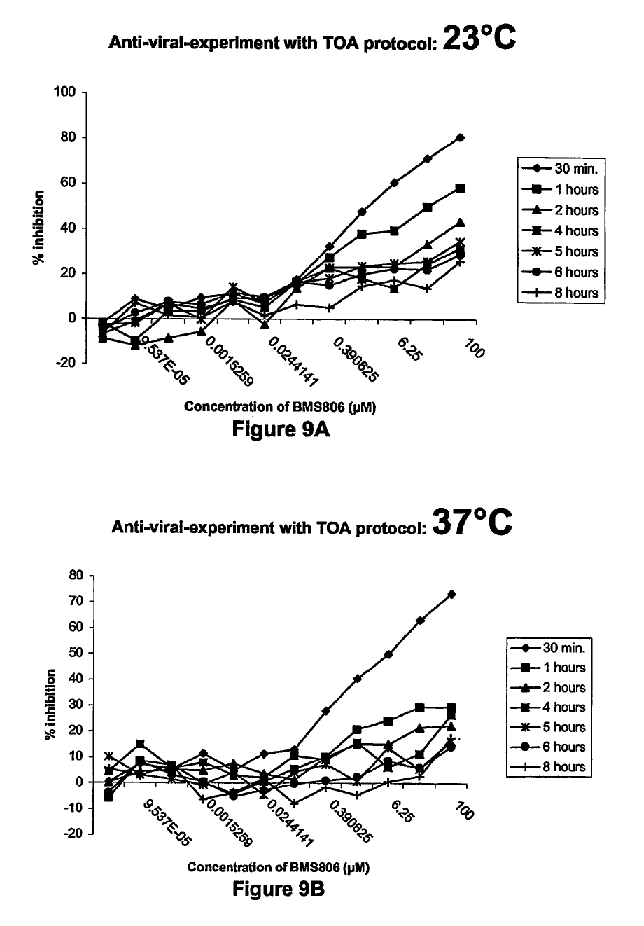

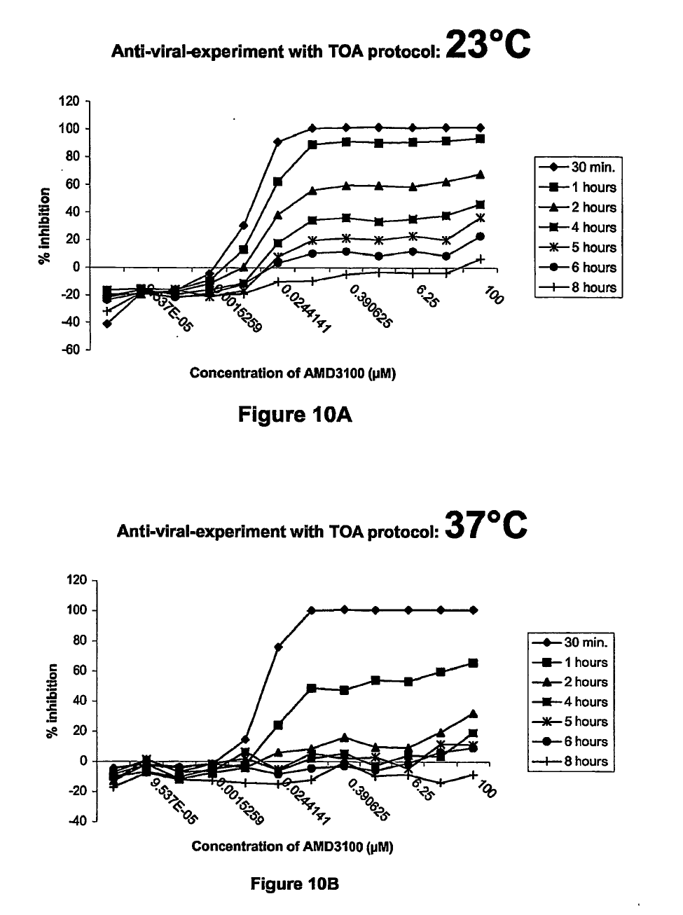

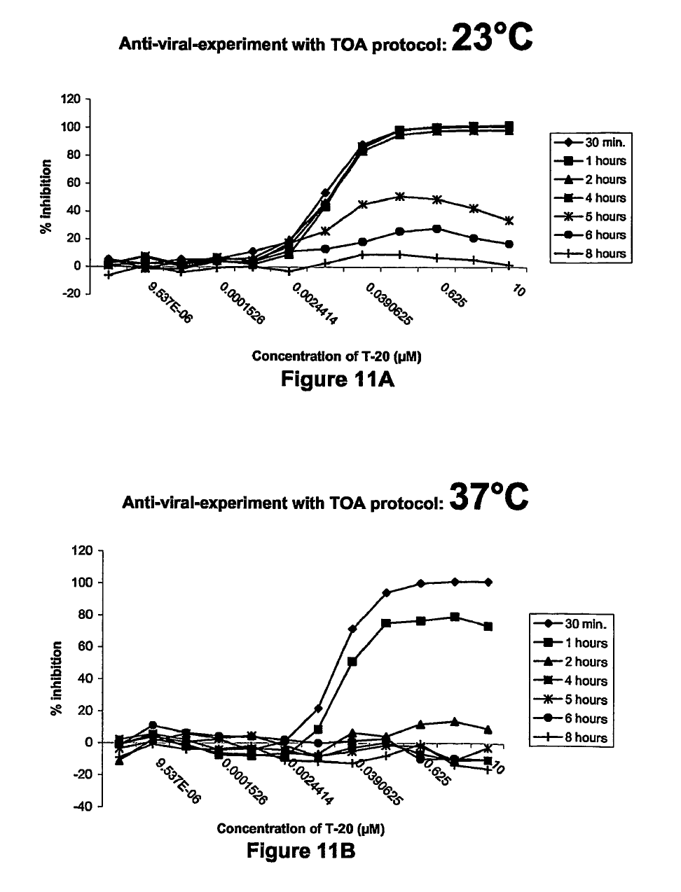

Time response curves for activity of tested compounds in 96-well plates and in 384-well plates are presented in FIG. 6 and FIG. 7, respectively. From the figures it can be seen that all reference compounds behaved as expected. Similar results are obtained for 96-well plates and 384-well plates. More in particular, it is clear that the entry inhibitors ATA, AMD3100 and T20 all demonstrated a strong inhibitory activity in the early stages of addition.

2.3 Visualization of TOA Experiment

The image shows that entry inhibitors acting on subsequent substages loose their activity at expected TOA points. Clearly, the next stage in the HIV-1 replication cycle (reverse transcriptase stage) can be distinguished from the entry stage.

Example 2 illustrates that the present method allows detecting compounds having an effect on the HIV entry process.

### Example 3

Experiments were performed according to the invention, where the effects of different inhibitors, at different concentrations and delta t's upon HIV-infected cells were examined at two different temperatures. Experimental detail is provided in the Figure legends 9 to 11.

The results demonstrate the effect of temperature upon the inhibitory activity of various HIV inhibitors, and show the clear advantage of the method of the invention.

1. A multi-well assay for identifying a compound inhibiting the replication cycle of a micro-organism comprising the subsequent steps of:
a) preparing a multi-well comprising micro-organism-coated host cells,
b) initiating at time t micro-organism infection and replication in said micro-organism-coated host cells such that micro-organism infection and replication is initiated synchronically in all host cells,
c) bringing at time t+Δt a candidate compound at one or more concentrations into contact with a part of the host cells,
d) repeating step c) after a time interval of Δt for another part of said host cells,
e) optionally repeating steps c) and d) using one or more other candidate compounds at one or more concentrations, and
f) determining whether said candidate compound has inhibited micro-organism replication in said host cells.

2. The assay according to claim 1 wherein said micro-organism is HIV.

3. The assay according to claim 1, whereby Δt is shorter than the time required for passing from one stage to another stage in the micro-organism replication cycle.

4. The assay according to claim 2, whereby Δt is shorter than the time required for passing from the entry stage to the reverse transcription stage in the micro-organism replication cycle.

5. The assay according to claim 2, whereby the compound is identified at inhibition of any one of the HIV entry steps: CD4 receptor attachment phase, co-receptor binding phase, and membrane fusion events.

6. The assay according to claim 1, wherein Δt, at which compounds are repeatedly added to the multi-well, comprises 1, 2, 3, 4, 5, 10, 15, 20, 25, 30, 40, 60, 120, 240 or 360 minutes.

7. The assay according claim 1, whereby steps c) to e) are performed under constant reaction conditions including under a CO₂-concentration of 5%, a relative humidity comprised between 95 and 100% and a temperature of 37° C.

8. The assay according to claim 1, whereby micro-organism replication in said host cells is initiated at time t by simultaneously bringing all cells at a temperature suitable for initiating micro-organism infection and replication.

9. The assay according to claim 1, whereby said multi-well is prepared by the steps of
a) coating host cells with a micro-organism at a high multiplicity of infection,
b) removing unadsorbed micro-organism, and
c) bringing said micro-organism-coated host cells onto said well.

10. The assay according to claim 1, whereby said host cells in said multi-well are able to express a gene encoding a detectable marker.

11. The assay according to claim 10, wherein a vector that expresses a gene encoding a detectable protein under the control of a HIV responsive promoter is introduced in said host cells.

12. The assay according to claim 1, wherein said micro-organism is labeled with a detectable protein.

13. The assay according to claim 1, whereby determination of said candidate compound is performed by detecting the presence or absence of said detectable marker.

14. The assay according to claim 13, wherein the presence or the absence of said detectable marker is detected by means of digital imaging techniques.

15. An apparatus for carrying out an assay according to claim 1, comprising:
a support suitable for supporting a multi-well comprising micro-organism-coated host cells, optionally moving in one or more directions,
one or more vials for containing a suspension of micro-organism,
one or more vials for containing one or more compounds,
pipetting means for dispensing micro-organisms in said multi-well, optionally moving in one or more directions,
pipetting means for dispensing one or more compounds in said multi-well, optionally moving in one or more directions,
pipetting controlling means for controlling dispensing by said pipetting means, and
environment controlling means for keeping conditions in said apparatus constant while bringing one or more compounds into contact with the host cells.

16. The apparatus according to claim 15 wherein said micro-organism is HIV.

17. The apparatus according to claim 15 further comprising a temperature-controlled multi-well support.

18. The apparatus according to claim 15 further comprising an insulating cover.

19. The apparatus according to claim 15 wherein the circuitry of pipetting and environment controlling means are sealed against high humidity.

20. Compounds identifiable with an assay according to claim 1.

21. Pharmaceutical composition comprising a therapeutically effective amount of one or more compounds identifiable with an assay according to claim 1 and a pharmaceutically acceptable excipient.

22. Use of a compound, identifiable with an assay according to claim 1, as a medicament.

23. Use of a compound, identifiable with an assay according to claim 1, for the manufacture of a medicament for treating infectious diseases.

24. A method of treating AIDS, comprising administering a therapeutically effective amount of a compound identifiable with an assay according to claim 2 to a patient in need thereof.
